(19) United States
(12) Patent Application Publication
(10) Pub. No.: US 2015/0119469 A1
(43) Pub. Date: Apr. 30, 2015

(54) Formulations and kit for biometric deposition of apatite on teeth

(71) Applicant: Heraeus Kulzer GmbH, Hanau (DE)

(72) Inventors: Susanne BUSCH, Neu Anspach (DE); Andreas UTTERODT, Neu Anspach (DE); Michael GERLACH, Hofheim (DE)

(21) Appl. No.: 14/481,127

(22) Filed: Sep. 9, 2014

(30) Foreign Application Priority Data
Sep. 9, 2013 (DE) 10 2013 109 847.9

(51) Int. Cl. A61C 19/06; A61D 5/00; A61K 6/033

(52) U.S. Cl. CPC A61C 19/063; A61K 6/033; A61D 5/00
USPC 514/769; 433/217.1; 433/1

(57) ABSTRACT

The invention proposes a kit and formulations for biomimetic deposition of apatite on teeth from a partially elastic shaped body comprising an envelope, whereby the shaped body a) comprises at least one mineralization matrix containing a gel that comprises water-soluble phosphates or phosphates that can be hydrolysed to form water-soluble phosphate ions and has a pH value of 2 to 8, optionally fluorides, and b) the at least one or a second mineralization matrix comprises a second gel having a pH value of 3.5 to 14 comprising calcium ions or compounds releasing calcium ions. Moreover, the method for producing the formulation and the use thereof for the deposition of apatite, in particular of needle-shaped fluorapatite crystallites, is claimed. Using the formulations according to the invention, it is feasible to deposit more than or equal to 1 μm apatite, in particular fluorapatite, on tooth surfaces in order to seal or brighten porous tooth surfaces.

The present application claims priority of German Patent Application No. 10 2013 109 847.9, filed Sep. 9, 2013.

The invention proposes a kit and formulations for biomimetic deposition of apatite on teeth from a partially elastic shaped body comprising at least a partial envelope (casing), whereby the shaped body a) comprises at least one mineralization matrix containing a gel that comprises water-soluble phosphates or phosphates that can be hydrolyzed to form water-soluble phosphate ions and has a pH value of 2 to 8, optionally fluorides, and b) the at least one or a second mineralization matrix comprises a second gel having a pH value of 3.5 to 14 comprising calcium ions or compounds releasing calcium ions. Moreover, the method for producing the formulation and the use thereof for the deposition of apatite, in particular of needle-shaped fluorapatite crystallites, is claimed. Using the formulations according to the invention, it is feasible to deposit more than or equal to 1 μm apatite, in particular fluorapatite, on tooth surfaces in order to seal or brighten porous tooth surfaces.

Teeth are hard biomaterials in the form of composites based on proteins and apatite comprising calcium and phosphate. Enamel, i.e. the outer layer of the crown of the tooth, is the hardest part of the tooth and contains no viable cells. It consists of inorganic crystals which typically are present in highly oriented arrangements. Enamel is a tissue, which, once it is produced, remains nearly unchanged for life since the cells involved in building up the teeth die as soon as tooth formation is completed. Finished enamel consists of approx. 95% by weight apatite, approx. 3% by weight proteins and lipids, and approx. 2% by weight water.

In order to prevent or repair tooth damage, in particular due to caries, attempts to use remineralizing systems have been made for a long time. These attempts initially involved the application of calcium phosphate compounds to improve the properties of the teeth.

Such one-component systems attempting to apply pre-made tooth substance, for example apatite, hydroxyapatite or other calcium phosphate compounds, to the teeth, are described, inter alia, in EP 0666730 B1 or WO 01/95863. It is a problem of said systems that treating the tooth substance with calcium phosphate compounds does not lead to the growth of an apatite that is structurally similar to the tooth substance, but rather to mere deposition of apatite crystals on the tooth substance, whereby the morphology of the apatite crystals is very different from that of tooth substance.

Accordingly, there is no strengthening effect on the enamel and no permanent filling of lesions, since the deposited apatite crystals do not comprise sufficient similarity and adhesion to the tooth substance.

Due to modern dietary habits, which often involve acidic food items, erosions of the dental hard substance that are not due to bacteria are on the rise [Dentale Erosionen: Von der Diagnose zur Therapie, Adrian Lussi, Thomas Jaeggi, Quintessenz Verlag].

But not only food items such as strongly acidified sweets, soft drinks or alcopops play a role in this regard, but the trend towards nutrition containing more fruit can also lead to dental problems. The continuous exposure to acids makes the enamel thinner and more porous. In extreme cases, the enamel can be dissolved totally and/or abraded such that the sensitive dentine is exposed. In the neck region of the teeth, in particular, which is protected by a very thin layer of enamel only, this occurs frequently. Acid-caused erosion can then proceed at even faster rates since dentine is more acid-soluble than enamel and wedge-shaped defects in the dental hard substance are often caused. Exposed dentine leads to sensitive, pain-sensitive teeth. However, sensitive dental necks can just as well be a consequence of inappropriate brushing habits. Increasing age is another reason for the enamel getting thinner. Habitual bruxism can also abrade the enamel at the incisal edges. Due to the improved prophylaxis in dentistry and strict addition of fluoride to most toothpastes and the availability of additional care products, caries is decreasing, but since the population in the industrialized countries is ageing and functional teeth have to work longer, the significance of non-cariogenic losses of dental hard substance is increasing as well.

Some forms of administration described to be suited for inducing the mineralization of apatite on the surface of teeth are known. U.S. Pat. No. 6,521,251 describes a composition that contains not only carbamide peroxide, but also calcium phosphates which are slightly more soluble than apatite, such as mono-, di- or tricalcium phosphate. But still, all these calcium phosphates are poorly water-soluble, such that the tooth cleaning means described are expected to have an abrasive rather than a remineralising effect. In fact, U.S. Pat. No. 5,851,514 describes, inter alia, the addition of dicalcium phosphate as an abrasive.

U.S. Pat. No. 6,419,905 mentions the addition of potassium salts (e.g. citrate) and fluoride to the peroxide. Fluoride is suited for binding, in particular, calcium and phosphate ions from the saliva, leading to the precipitation of fluorapatite. If no other ions are added, the formation of CaF₂ has also been observed. Calcium fluoride particles can be stored in the plaque and can release fluoride for extended periods of time since they are more soluble than the apatite of the hard dental substance. However, conscientious repeated daily cleaning of the teeth largely removes the plaque. Accordingly, the effect of calcium fluoride is short-lived and the fluoride-containing products need to be applied in regular intervals. No formation of new apatite has been observed with products of this type.

Patent JP20000051804 describes the concurrent use of concentrated phosphoric acid, conc. H₂O₂ and fluorapatite powder. The use of concentrated phosphoric acid in this context appears questionable as this substance can dissolve healthy enamel to a notable degree. Moreover, the bleaching solution is strongly irritating and must not contact the gingiva, although this is true, at a lesser level, of all tooth-bleaching agents having an oxidative effect. Moreover, repeated application does not lead to the build-up of a mineralization layer.

An acid-free application is described in U.S. 20050281759. Calcium peroxophosphate is proposed as essential ingredient in this context. The underlying rationale being that a single substance is to have the brightening and remineralising effect, since the release of calcium and phosphate ions is triggered parallel to the oxidation. It is not clear whether or not the salts can attain any significant build-up of apatite during their relatively short period of action. U.S. Pat. No. 6,303,104 describes an oxidant-free two-component system consisting of soluble calcium and phosphate salts, which is claimed to have a brightening effect as well. The brightening is said to be caused by the addition of sodium gluconate, which forms complexes with staining metal ions (e.g. iron) from the enamel. Mixing of the components is expected to immediately lead to precipitation of the poorly-soluble calcium phosphates and it is not obvious why there should be pronounced remineralization, even more so as the product is a toothpaste to which the tooth surfaces are exposed for no more than a few minutes at a time. U.S. Pat. No. 6,102,050 describes a dental floss having titanium dioxide particles that is said to have a brightening, remineralizing, and desensitizing effect on the interdental surfaces. Titanium dioxide microparticles of a size of 0.1 to 1.5 μm are to act both as a mild abrasive and are to be absorbed by the enamel, which is said to be associated with a brightening effect. Presumably, the particles can no more than get incorporated mechanically into suitable hollow spaces which does not promise to lead to stable anchoring and can have no more than a temporary effect.

All patents described thus far fail to take into consideration that bio-minerals attain their high degree of structural organisation and stability only because they are formed in the presence of specific biomolecules that define the formation of the micro- and macro-structure.

WO 2005/027863 describes a tooth care product that is said to possess cleaning, remineralizing, desensitizing, and brightening effects. The nano-scale apatite-gelatine composite proposed as active component for remineralization and brightening precipitates in the presence of an aqueous gelatine solution and thus has polypeptides incorporated into it. This material is said to form a protective layer of dentine-like structure on the surface of the tooth due to so-called “neo-mineralization”, whereby the protective film is said to smoothen the surface and to be able to seal open dentine tubules. This effect is not comprehensible for a toothpaste, since said tooth care product preferably contains only 0.01-2% by weight “nanite” (WO 01/01930). The active substances can act for no more than a few minutes daily. Any pronounced or surface-covering deposition of mineral is doubtful. Moreover, no deposition of mineral on enamel is described. No continuous increase in the thickness of the film upon extended application of the care product is described either. Moreover, the porous, poorly ordered structure of dentine is not capable of protecting the tooth from corrosive attacks. The commercially available product,

Tooth Mousse or Mi-Paste, is based on patent specifications by Reynolds [WO 98/40406] and is said to remineralize porous enamel. The invention is based on casein (CPP) having a stabilizing effect on amorphous calcium phosphate (ACP). In contact with the hard dental substance, the CPP-ACP agent is to remineralize into hydroxylapatite. A protective film of dentine-like structure of this type appears unsuited to provide long-term protection.

It is common to all patents that they only refer to remineralization without documenting same and/or without having a desensitizing effect. US 2012/0027829A1 describes the formation of hydroxylapatite layers (HAP) on dentine by repeatedly applying pasty mixtures of propylene glycol, glycerol, xylitol, polyethylene glycol, cetylpyridiniumchloride, tetracalcium phosphate, and an alkali salt of phosphoric acid to the teeth. Since tetracalcium phosphate reacts immediately with phosphoric acid salts in the presence of water, two separate pastes are produced first and mixed only right before application. No formation of HAP on enamel is described and no data is provided on the layer formed, which also was not reproducible in own experiments.

The technique described in US2005220724 and DE 102004054584.7 provides a fluorapatite layer which possesses enamel-like strength and increases in thickness upon repeated application. Water-soluble phosphate and fluoride salts are incorporated in the buffered gel A, whereas calcium ions are incorporated in gel B. Optionally separated through an ion-free protective layer, the gelatine gels, which are solid at physiological temperature, are applied, while heating, to the tooth surfaces one after the other. An increase of the thickness of the layer as a function of the exchange cycles of the gels can be observed. The growth rates are 0.5 to 5.0 μm/day. The biological structures of the tooth substance are replicated individually by the fluorapatite; hollow spaces due to open dentine tubuli are sealed after a few exchange cycles.

Regarding the use in humans, it is inconvenient that the gels need to be heated before application. The application of the second and third gel layer may cause underlying, previously applied gel layers to liquefy again and mix with the upper layers in undesirable manner. Small amounts applied as described, in particular, dry out quickly upon exposure to air and are then difficult to liquefy by heating them. The method hardly allows exactly defined amounts of gel of even thickness to be applied to the tooth. Moreover, the three gel layers, each being up to 6 mm in thickness, are quite bulky, which leads to problems in the case of protective systems, such as splints or plasters, as space for large gel reservoirs needs to be created in this case.

Moreover, the method becomes increasingly elaborate when the entire jaw including all tooth surfaces is to be treated. Since an application period for the formation of fluorapatite should not be less than 8 hours under ideal conditions, it would be of advantage if the patient could use the system himself/herself by using it before going to bed. For this, the patient would have to warm up the gels and place them precisely on the teeth, which is very difficult since warmed-up liquid gelatine is very tacky. Moreover, this is associated with a major risk of burns. It is also disadvantageous that the gels stay liquid and do not safely adhere on the teeth in the oral environment.

Since the gels leak despite the presence of protection, such as, e.g., an individualized deep-drawing splint, the splint needs to be sealed with a suitable sealing system, which renders the method even more complicated.

The use of pre-made gel strips in DE 10 2006 055 223 A1 is advantageous in that there is no cumbersome heating involved and the strips are of the same thickness. However, one major disadvantage is that the strips reach only partial regions of the teeth. However, since erosions basically affect all surfaces of teeth, it would be desirable to have the mineralization kit exert its effect in all places. Moreover, it is very cumbersome to unpack the two strips and to insert them, for example, into a deep-drawing splint, which, in addition, also needs to have a reservoir for the gel strips. The issue of sealing the splints is not solved satisfactorily. Since the strips liquefy in the oral environment, sealing is required though in order to prevent the agents from leaking into the oral space.

The invention describes a method based on patents US2012195941(A1) and US2008241797 (A1) that can be used to apply an enamel-like fluorapatite layer onto teeth. In the embodiment described, two mineralization strips are formed appropriately such that individual tooth surfaces, a group of teeth or multiple tooth surfaces can be treated at once. It is also feasible to treat tooth surfaces of entire jaws.

The invention is based on the object to be able to provide a mineralization kit that produces, highly reproducibly, high-quality enamel-like coatings made of apatite on hard dental substance aiming to protect the hard dental substance from excessive loss of enamel. The ingredients are to be much like the biological original and the application should be as simple as possible. Further objects were to simplify the application significantly; preferably, the application should be possible both at the dentist's and directly by the user. Moreover, the application time of a single use of a kit was to be increased. One object was to solve the issue of sealing of the gels and to provide a formulation, which preferably needs no separate sealing without preventing the deposition of the apatite.

The objects are solved through a formulation and a kit as described hereinbelow as well as through the methods for producing the formulation as also described hereinbelow.

The objects were met in that the mineralization matrix composed on the basis of denatured collagen or other gel-forming agents and mineral substances is made insoluble in the oral environment through chemical modification, in particular as a formulation having separate shaped bodies (form bodies) or as a multi-part shaped body each comprising at least one or two mineralization matrices. Surprisingly, the mineralization activity is affected beneficially despite the modification, since the gel no longer spreads in the oral environment and can act during the entire duration of treatment. Said chemical modification preferably proceeds by means of at least partially chemical cross-linking at the surface of the mineralization matrix such that at least one partially chemically cross-linked plane or partial envelope (casing) is formed. Preferably, the at least one plane is a surface of the mineralization matrix; preferably, the planes form an outer envelope at the surfaces of the mineralization matrices. According to the invention, the cross-linked planes or the envelope act like a membrane through which aqueous media, such as saliva, can penetrate into the mineralization matrix and, concurrently, apatite can be deposited on the tooth surfaces outside of the mineralization matrix, which contact the at least one plane or the envelope of the mineralization matrix. According to the invention, the plane or envelope formed can, on the one hand, adapt optimally to the surfaces of the teeth when the material swells in the mouth due to it being a very thin layer and, on the other hand, also favours the deposition of apatite at the approximal spaces. Moreover, the mineralization matrix adapts optimally to the contour of the tooth at body temperature. It is another particular advantage of the cross-linking at the surface of the mineralization matrix, during which a shaped body is formed, that an at least partially elastic shaped body is thus made which can adapt optimally to the surface contour of teeth and rows of teeth. Due to swelling of the mineralization matrix in the oral environment, the partially elastic shaped body adapts particularly well to the tooth surfaces. In order to affix the shaped body, comprising at least one mineralization matrix, optimally to the buccal, labial, mesial and/or approximal surfaces of the teeth, it is advantageous to place and/or to provide the at least one shaped body in a dental splint. Since the shaped body according to the invention is affixed with a dental splint, apatite can be deposited, at least on part or almost completely, on the teeth of the upper and lower jaw in buccal, mesial, labial, palatinal, distal and approximal position. The at least one shaped body according to the invention allows, easily and for the first time, for biomimetic remineralization of at least, partially, more than or equal to 1 μm, preferably of more than or equal to 2 μm, preferably of more than or equal to 3 μm through inserting the shaped bodies into a splint and placing the splint onto the teeth over night, for example for approx. 8 to 12 hours, optionally up to 16 or 24 hours, alternatively during the day as well, whereby biomimetic remineralization of at least partially, preferably on average, from 1 to 10 μm, 1 to 5 μm is particularly preferred. It is particularly preferred for the remineralization to occur in two-dimensional manner in an area and with the thickness of the layer formed being as homogeneous as possible.

Surprisingly, it has been found that the mineralization product forms more evenly and more densely on the tooth surface after the chemical stabilization. The cross-linked plane or envelope is so porous that comparable apatite layers can be deposited despite the formation of the envelope, as is shown in examples 2 to 4 and 6 according to the invention by comparison to examples 1 and 5. Presumably, the chemical modification leads to the formation of a less temperature-sensitive and preferably less hydrolysis-sensitive network of polypeptides around the mineralization matrix, whose pores are large enough, though, to still allow molecules and ions forming the stable, ordered apatite layer on the tooth surface to pass. The chemically cross-linked layer or plane basically acts like an ion- and molecule-permeable membrane.

A sufficiently thick, homogeneous, and stable apatite layer can be attained by the optimal interplay of the components: mineral salts, buffer, and pH value. It is crucial to select the concentrations correctly at a corresponding degree of cross-linking. The formulation according to the invention can be used for depositing apatite on teeth or any other biological surfaces, such as a bone matrix.

Since the application of the formulations according to the invention is simpler, the system can be used both by dentists on patients and by the patients on themselves. The deposition of a fluoride-rich calcium phosphate layer can reduce sensitivities on the teeth, it can reduce cracks and initial porosities and increases the acid stability of the teeth. Initial losses of hard dental substance due to acid erosion can be stopped and/or partially or completely reversed. The increased fluoride content in the layers as compared to untreated teeth reduces the solubility of the newly formed mineral. The natural enamel is protected from toothbrush erosion by the protective layer.

The object of the invention is a formulation that is well-suited for the deposition of apatite, in particular well-suited for biomimetic deposition of apatite, selected from fluorapatite (Ca₅[FRPO₄)₃), hydroxylapatite (Ca₅[FRPO₄)₃) or mixtures thereof on teeth or on a bone matrix of vertebrates, whereby the formulation comprises at least one partially elastic shaped body, in particular three-dimensional, preferably flat shaped body, comprising at least one mineralization matrix containing at least one gel, whereby the shaped body comprises, at least in part, in at least one plane a reduced solubility with respect to aqueous media as compared to the mineralization matrix, whereby the plane acts as membrane, and
a) the at least one mineralization matrix comprises a gel containing water-soluble phosphates or phosphates that can be hydrolysed to form water-soluble phosphate ions and has a pH value of 2 to 8 (phosphate component), and
b) the at least one or a second mineralization matrix comprises a second gel comprising calcium ions, in particular compounds containing water-soluble calcium ions, or compounds releasing calcium ions (calcium component) having a pH value of 3.5 to 14, comprising, comprising calcium ions or compounds releasing calcium ions (calcium component), particularly preferably the at least one mineralization matrix is present in a first shaped body I. and the second mineralization matrix is present in a second shaped body II., alternatively two mineralization matrices are present in one shaped body.

### BRIEF DESCRIPTION OF THE DRAWINGS

The invention will now be described in greater detail with reference to the drawings, wherein:

FIG. 1 shows the typical surface morphology of the coating at the boundary between coated and uncoated sample after one treatment.

FIG. 2 shows a SEM image of the boundary between coated enamel and uncoated enamel).

FIG. 3 shows a typical layer after one application of the mineralization kit and staining with 0.1% rhodamine solution.

FIG. 4 shows the typical surface morphology of the coating at the boundary between coated and uncoated sample after one treatment.

FIG. 5 shows a detail of the upper edge of the tooth including the fluoride-rich layer.

FIG. 6 shows 3× tooth discs treated on half of a side before (left) and after toothbrush abrasion.

FIG. 7 shows 3× tooth discs treated on half of a side before (left) and after toothbrush abrasion.

FIG. 8 shows dentine surface growth.

FIGS. 9A and 9B disclose general embodiments of the shaped bodies according to the invention.

According to a preferred embodiment of the invention, the at least one mineralization matrix or the two mineralization matrices each independently comprising at least one gel are present in the form of a flat element e.g. two-dimensional element, element of area, or of an at least partial negative image of the jaw. It is also preferred that the at least one or two partially elastic shaped body/bodies is/are also present in the form of a flat element or of an at least partial negative image of the jaw.

It is preferred that the aforementioned at least one plane is a plane arranged on the outer surface or is an essentially complete outer envelope.

Formulations according to the invention comprise one or two shaped bodies in the form of a flat element each having one enclosing envelope having reduced solubility that functions, in particular, as a membrane. In an alternative embodiment, the formulation comprises a shaped body in the form of a flat element each having an enclosing envelope having reduced solubility that functions, in particular, as a membrane, having two mineralization matrices situated one on top of the other each comprising one gel, whereby one gel comprises the phosphate component and one gel comprises the calcium component. Said two mineralization matrices are comprised by the shaped bodies A and B of the kit. According to another alternative embodiment, a flat element comprises a mineralization matrix having a gel, whereby the gel optionally is sub-divided by a membrane layer into two reservoirs, one comprising the phosphate component and one comprising the calcium component. The aforementioned shaped bodies are preferably present as flat element with a layer thickness of 5 to 6,000 μm, whereby mineralization matrices in the form of a flat element each having a layer thickness of 5 to 3,000 μm are particularly preferred, preferably 100 to 600 μm, in particular 300 to 600 μm or about 500 μm plus/minus 200 μm, in particular plus/minus 100 μm, in particular plus/minus 50 μm. The shaped bodies according to the invention comprise, at least partially, an envelope or at least one plane of reduced solubility. Instead of a flat element, the shaped body can just as well be present in the form of at least a partial negative image of the jaw, preferably as a negative mould of the teeth of the upper and/or lower jaw. Flat elements of the calcium component are preferably present having a layer thickness of 500 to 1,500 μm, particularly preferable are mineralization matrices in the form of a flat element each having a layer thickness of 500 to 1,200 μm or about 1,000 μm plus/minus 100 μm, in particular plus/minus 50 μm. Flat elements of the phosphate component are preferably present having a layer thickness of 100 to 1,000 μm, particularly preferable are mineralization matrices in the form of a flat element each having a layer thickness of 150 to 800 μm, preferably 300 to 800 μm, in particular 400 to 600 μm or about 500 μm plus/minus 200 μm, in particular plus/minus 100 μm, in particular plus/minus 50 μm.

Formulations according to the invention have a water content after cross-linking, and optionally after subsequent drying, of 8 to 60% by weight, preferably of 30 to 55% by weight. It is also preferred for the gels of the formulations according to the invention to have a water content after cross-linking, and optionally after subsequent drying, in the mineralization matrix of the phosphate component of 20 to 40% by weight, preferably of 25 to 35% by weight, particularly preferably about 30% by weight with a deviation of plus/minus 5% by weight. Accordingly, formulations of the calcium component that have a water content after cross-linking, and optionally after subsequent drying, in the mineralization matrix of 30 to 60% by weight, preferably of 40 to 60% by weight, more preferably about 50% by weight with a deviation of plus/minus 5% by weight are preferred. The formulations thus produced can subsequently be welded into blisters, sachets or the like such as to be air- and moisture-tight.

Preferably, the following alternatives of the shaped bodies can be present: a) two shaped bodies each having a partial, preferably essentially complete, envelope, particularly preferably having an enclosing envelope, preferably two shaped bodies with a cross-linking of the upper and/or lower side of the shaped bodies that are present as flat element, b) a shaped body having two mineralization matrices, preferably separated through a membrane, c) a shaped body having a mineralization matrix comprising two gel regions that are optionally separated through a membrane. Using two mineralization matrices or two gel regions for the phosphate component allows a concentration gradient to be adjusted in the shaped body. Accordingly, a concentration profile can also be adjusted for a shaped body containing the calcium component. The envelope or the plane comprises reduced solubility with respect to aqueous media as compared to the mineralization matrix, preferably the envelope functions as a membrane and reduces the dissolution of the mineralization matrix in the oral environment. However, H₂O from saliva can penetrate and fluorides, calcium ions, and phosphate ions as well as composites or polypeptides can diffuse through the envelope and can be deposited on the tooth surface as apatite, hydroxylapatite or fluorapatite. The envelope encloses a water-rich gelatine matrix from which the composites, ion-loaded waterl molecules and/or hydrated ions can diffuse. The diffusion of the composites is important since the composites are organic macromolecule-substituted hydroxyapatites that form the enamel, and in order to deposit these on the tooth surfaces. According to the invention, fluorapatite-protein composites from the shaped bodies are deposited on the tooth surfaces.

According to the invention, the at least one plane or the envelope form the outer boundary of at least one mineralization matrix. The plane or envelope can be obtained in a variety of ways by chemically cross-linking the mineralization matrix or by applying a coating to the mineralization matrix in order to form a mineralization matrix surface that is permeable for ions and water and acts as membrane. The cross-linking or coating can be effected by immersing, application techniques, such as painting, spraying, rolling, and other measures known to a person skilled in the art. The planes or the envelope can just as well be formed to have irregular or regular perforations and/or pores or pore-forming agents can be added.

The fluorapatite deposited from the formulation according to the invention is preferably present on the teeth in crystalline form, preferably micro-crystalline, particularly preferably in the form of needle-shaped crystallites. The at least partial apatite layer, preferably contiguous apatite layer, deposited on the tooth surfaces in the course of one application cycle of approx. 8 hours has a layer thickness of at least 1 μm, in particular 2 μm. The scope of the invention also includes at least partially non-contiguous apatite layers that cover at least a part of the treated tooth surface irregularly to preferably homogeneously. The apatite layers can, at least in part, be up to 15 μm, preferably essentially homogeneous, which is preferred, apatite layers of more than or equal to 2 μm, more preferably more than or equal to 5 μm to 13 μm, and on average of 2 to 10 μm are obtained.

Reduced solubility of the chemically cross-linked plane or envelope with respect to aqueous media as compared to the mineralization matrix shall be understood as follows: the mineralization matrix not chemically cross-linked and, optionally, the mineralization matrix modified with a plasticizer only, for example the gelatine cross-linked to glycerol as adduct via hydrogen bonds. It is preferable to use the following gelatine qualities: Bloom 175 to 300 or higher with Gelatine Bloom 300 (pork rind) being preferred.

Teeth of vertebrates include human teeth, prostheses of human teeth, deciduous teeth (Dentes decidui), permanent teeth (Dentes permanentes), crowns, inlays, implants, teeth of animals, such as domestic and livestock animals, such as dogs, horses, cats.

In the scope of the invention, an at least partially elastic shaped body shall be understood to mean a three-dimensional shaped body that is present as flat element or at any three-dimensional geometry, in particular in the form of an at least partial negative image of the jaw, and has elastic properties. The shaped body shall be considered to be elastic or partially elastic if the body changes its shape when exposed to a force and returns to its original shape, partly or fully, when the force ceases to act on it. The shaped body preferably possesses the property of being elastic or partially elastic when it is applied in the oral environment and preferably after production. The elasticity may decrease upon excessive drying.

In a further formulation that is particularly preferred according to the invention, the mineralization matrix comprises in a) the following composition: a) the at least one mineralization matrix comprises a gel comprising (i) water-soluble phosphates or phosphates that can be hydrolyzed to form water-soluble phosphate ions, in particular Na₂HPO₄, whereby the phosphate content in the mineralization matrix preferably is 1 to 10% by weight, more preferably 2 to 8% by weight, particularly preferably 5 to 8% by weight, (ii) a content of water or of a mixture of water and an organic solvent, (iii), optionally, at least one carboxylic acid, in particular a hydroxycarboxylic acid, such as lactic acid, and/or a buffer system, in particular a buffer system is present for adjusting the pH value in the range of 2 to 8, in particular from 3.5 to 8, preferably from 3.5 to 6, particularly preferably about 5.5 plus/minus 0.5. The content refers to PO₄³

Concurrently, the formulation that is particularly preferred according to the invention comprises a mineralization matrix in b) of the following composition: the second mineralization matrix or the at least one mineralization matrix comprises a second gel comprising (i) calcium ions or compounds releasing calcium ions, in particular calcium dichloride or hydrates thereof, preferably in addition calcium sulfate, nanoapatite, sodium carbonate or calcium oxalate, whereby the calcium content in the mineralization matrix preferably is 1 to 10% by weight, more preferably more than or equal to 1.5 to 7.5% by weight, (ii), optionally, water or a mixture of water and an organic solvent, and (iii), optionally, at least one carboxylic acid, such as a hydroxycarboxylic acid, for example lactic acid, and/or a buffer system. It is preferred to use fruit acids and alkali salts to produce the buffers. The content refers to calcium (Ca²⁺).

Moreover, it is preferred that the formulation comprises, in the at least one mineralization matrix a gel that comprises at least one water-soluble fluoride (F-), with fluoride ions or a compound releasing fluorides. Particularly preferably, the formulation in a) comprises as further component (iv) at least one water-soluble fluoride or one compound releasing fluorides.

According to a preferred refinement of the invention, the at least one water-soluble fluoride or the at least one compound releasing fluorides comprises, in particular in a), the at least one mineralization matrix, (i) at least one non-substituted or substituted alkyl groups-comprising quaternary mono- or poly-ammonium compound, preferably having four substituted alkyl groups, whereby the at least one substituted alkyl group comprises hydroxyalkyl, carboxyalkyl, aminoalkyl groups having 1 to 25 C-atoms or organo-functional, hetero atom-interrupted groups having up to 50 C-atoms. Preferred ammonium compounds can contain 1 to 20 quaternary ammonium functions, preferably 1, 2, 3, 4, 5, 6, 7, 8 ammonium functions; it is preferable to use Olaflur (N,N,N′-tris(2-hydroxyethyl)-N′-octadecyl-1,3-diaminopropan dihydrofluoride) as water-soluble fluoride. Also preferred are amine fluorides, such as Oleaflur (C₂₂H₄₅FNO₂), Decaflur (9-Octadecenylaminhydrofluoride), ethanolamine hydrofluoride, ii) a fluorides-releasing organo-functional amino compound or a fluorides-releasing antiseptic based on organo-functional amino compounds, such as, in particular, fluorides of N-octyl-1-[10-(4-octyliminopyridin-1-yl)decyl]pyridin-4-imine, cetylpyridinium fluoride, or c) water-soluble inorganic fluorides, such as alkali fluorides, sodium fluoride, potassium fluoride, tin fluoride, ammonium fluoride, or fluorides-releasing inorganic fluorides, such as zinc fluoride, zinc hydroxyfluoride.

Preferably, at least one gelforming agent selected from denatured collagen, hydrocolloids, polypeptides, proteinhydrolysis products, synthetic polyamino acids, polysaccharides, polyacrylates (Superabsorber) or mixtures comprising at least two of the aforementioned gel-forming agents can be present in the formulation as gel according to the invention. The polysaccharides and/or polyacrylates can preferably also be used, in particular, as second mineralization matrix and/or for formation of the at least one plane or envelope. It is preferable to use gelatine in the first mineralization matrix to which, according to the invention, a plasticizer such as glycerol or another polyol is added. Adding the plasticiser improves the handling properties of the gelatine.

In a formulation according to the invention, the mineralization matrix comprises, as gel, gelatine and preferably a plasticizer, preferably a polyol, such as glycerol and/or conversion products thereof, optionally in the presence of water. Alternatively, gelatine and a plasticizer such as sorbitol can be used just as well. The effect of the plasticizer is to increase the melting range by forming intermolecular hydrogen bonds. According to the invention, gelatine (denatured collagen, animal protein, protein) is preferably used as gel and acid-hydrolyzed collagen is used particularly preferably or gelatine and a polyol such as glycerol. Alternatively, casein, starch flour, cellulose, HPMC, gum arabic, galactomannanes, guar gum, konjac, xanthane, calcium alginate, dextrane, scleroglucan, pectin, carrageenan (K-, I- and A carrageenan), Agar-Agar, alginate, alginic acid, sodium alginate, calcium alginate, tragacanth can be used as gel, whereby gelatine or mixtures containing gelatine are preferred.

The core of the invention are formulations having at least one partially elastic shaped body, whereby the shaped body corresponds to at least one mineralization matrix containing at least one gel, whereby the mineralization matrix comprises, at least in part, in at least one at least partially chemically cross-linked plane, in particular covalently cross-linked plane, a reduced solubility with respect to aqueous media as compared to the corresponding mineralization matrix that is not chemically cross-linked in this way, in which just intermolecular hydrogen bonds form due to the presence of glycerol in the gelatine, whereby the at least partially cross-linked plane acts, in particular, as membrane, with preferably all outer surfaces of the mineralization matrix being chemically cross-linked, at least in part. For use on teeth, it is preferred that the mineralization matrix and the shaped body are present as a flat element or at least partial negative image of a jaw. A flat element according to the invention can just as well reproduce a surface texture that simulates the tooth surfaces of a dental arch.

According to an embodiment of the invention, the formulation comprises at least one mineralization matrix comprising at least one gel in at least one partially elastic shaped body, in particular two-dimensional shaped body e.g. flat or board shaped body, whereby the mineralization matrix is present in the form of a flat element and whereby the shaped body comprises at least two planes arranged on the outer surface or an outer envelope which each comprise a reduced solubility with respect to aqueous media as compared to the mineralization matrix, and which act, in particular, as membrane for chemical compounds such as the composites, for example made of polypeptides and salts, as well as for salts and water. It is reasonable in said embodiment that the partially elastic shaped body also is present in the form of a flat element. In this refinement, the formulation is particularly well-suited treating multiple teeth. The shaped body can just as well be present as at least partial negative image of the jaw.

Compounds for thermal stabilisation of the gel preferably comprise plasticisers based on polyols. Due to the enveloping of the matrices being as complete as possible, as is made feasible according to the invention, the addition of plasticisers is dispensable or a different excipient improving the handling properties of the gel can be added, depending on the application on hand. For example if just one tooth or one cavity in a tooth is provided with a shaped body with envelope, preferably in the form of a flat element or at least partial negative image of a jaw, sphere, granulate grain or capsule for apatite deposition and if the regions of the gels within the shaped body are present optionally separated by a membrane. The di- or poly-functional cross-linkers, preferably glutardialdehyde, are used as compounds for chemical cross-linking, in particular covalent cross-clinking, in the at least one plane or for forming the envelope of the mineralization matrix.

The chemical cross-linkers form covalent cross-linking sites with the gels, in particular with the polypeptides or polyamino acids. Preferably, the di- or poly-functional cross-linkers comprise dialdehydes, polyepoxides and/or polyisocyanates as well as mixtures comprising at least two cross-linkers. Furthermore, it is preferred to use pharmacologically tolerable cross-linkers. Preferred dialdehydes comprise alpha, omega dialdehydes of hydrocarbons, in particular comprising 2 to 50 C-atoms, in particular 4 to 10 C-atoms in the di-functional alkylene group. Treating the mineralization matrix with a cross-linker reduces the solubility of the gelatine to a level such that it does not liquefy in the oral environment for approximately 8 hours. Preferably, the treatment with glutardialdehyde proceeds for at least 5 s, depending on the application on hand the cross-linking may proceed for longer and thus be more pronounced, for example if a mineralization matrix is to remain in the oral environment for 12 to 16 hours. After rinsing for 40 s in an 0.5% glutardialdehyde solution, the solubility of the gel is reduced to a level such that it does not liquefy in the oral environment for up to 8 hours. The membrane (layers) that can be used optionally are free of ions. Instead of rinsing, immersing or spraying the solution onto the matrix are feasible just as well.

The cross-linker solution preferably has a cross-linker content of approx. 0.25 to 0.5% by weight, preferably of glutardialdehyde. It has been evident that the best results in terms of sufficient cross-linking and optimal permeability for the apatite composites to be deposited are obtained with a treatment time of 0 to 60 s, preferably approx. 5 to 40 s, particularly preferably 10 to 30 s, according to the invention about 20 seconds (s).

According to a preferred embodiment, the formulation comprises (I) a shaped body in the form of a flat element having at least two mineralization matrices that are optionally separated by a membrane (layer), each in the form of a flat element containing the gel with the following layer structure in the shaped body:
A) a first mineralization matrix in the form of a flat element comprising gel and water-soluble phosphates or phosphates that can be hydrolysed to form water-soluble phosphate ions, water-soluble fluorides or a compound releasing fluorides, water or a mixture of water and an organic solvent, optionally at least one carboxylic acid and/or a buffer system;
B) optionally membrane (layer);
C) a second mineralization matrix in the form of a flat element comprising gel and calcium ions or compounds releasing calcium ions, optionally water or a mixture of water and an organic solvent, optionally at least one carboxylic acid and/or a buffer system, whereby (I) the shaped body in the form of a flat element comprises at least one or two planes that are arranged up to a polygon on the outer surface or comprises an outer envelope which (a) can be obtained, at least in part, by chemical cross-linking or (b) correspond to a coating, and which act, in particular, as membrane, and comprise a reduced solubility with respect to aqueous media as compared to the mineralization matrices, or,

according to the invention, the formulation comprises (II) two separate shaped bodies each independently in the form of a flat element having at least one mineralization matrix, optionally separated by a membrane (layer), in the form of a flat element containing the gel, whereby the first shaped body comprises a first mineralization matrix in the form of a flat element comprising gel and phosphates, such as hydrogen phosphates, or phosphates that can be hydrolyzed to form water-soluble phosphate ions, water-soluble fluorides or compound releasing fluorides, water or a mixture of water and an organic solvent, optionally at least one carboxylic acid and/or a buffer system, optionally membrane (layer); and the

second shaped body comprises a second mineralization matrix in the form of a flat element comprising gel and calcium ions or compounds releasing calcium ions, optionally water or a mixture of water and an organic solvent, optionally at least one carboxylic acid and/or a buffer system;

whereby (II) the shaped bodies each comprise at least one or two to up to six planes that are arranged on the outer surface or an outer envelope, which (a) can be obtained, at least in part, by chemical cross-linking or (b) correspond to a coating, and which act, in particular, as membrane and comprise a reduced solubility with respect to aqueous media as compared to the mineralization matrices.

Also a subject matter of the invention are formulations comprising (I) a shaped body in the form of a flat element having at least two mineralization matrices, optionally separated by a membrane, each independently in the form of a flat element containing the gel at a layer thickness of 50 to 6,000 μm, in particular of 500 to 2,000 μm, preferably of 1,000 to 2,000 μm or 500 to 1,500 μm, or (II) two separate shaped bodies each independently in the form of a flat element each having at least one mineralization matrix in the form of a flat element containing the gel, whereby each shaped body independently has a layer thickness of 10 to 3,000 μm, preferably 50 to 1,000 μm, particularly preferably 100 to 750 μm, even more preferably 300 to 600 μm, yet more preferably about 500 μm plus/minus 300 μm, whereby the first shaped body comprising phosphates, such as hydrogen phosphates, or phosphates that can be hydrolysed to form water-soluble phosphate ions, has a layer thickness of 50 to 3,000 μm, in particular 100 to 3,000 μm, preferably of 300 to 1,000 μm, particularly preferably of about 500 μm plus/minus 200 μm or +/−50 μm, and/or the second shaped body comprising calcium ions or compounds releasing calcium ions has a layer thickness of 10 to 3,000 μm, in particular 100 to 3,000 μm, preferably of 300 to 1,000 μm, more preferably of 300 to 750 μm, even more preferably of about 500 μm plus/minus 200 μm or +/−50 μm.

Alternatively, the aforementioned shaped bodies are part of an at least partial negative image of a jaw with a corresponding multi-layered internal design that is to be arranged on the teeth.

The carboxylic acids are preferably selected from fruit acids, such as α-hydroxycarboxylic acids such as malic acid, citric acid, glycolic acid, lactic acid, and tartaric acid; amino acids, fatty acids, hydroxycarboxylic acids, dicarboxylic acids, and mixtures comprising at least two of the aforementioned acids and/or the buffer system comprises carboxylates of alkylcarboxylic acids, fatty acids, fruit acids, fumarates, amino acids, hydroxycarboxylic acids, dicarboxylic acids, and mixtures comprising at least two of the aforementioned acids or phosphate buffer. It is advantageous to use alkali and/or alkaline earth salts or zinc salts for the buffer systems.

The buffer systems comprise EDTA, TRIS: tris(hydroxymethyl)-aminomethane for pH 7.2 to 9.0, HEPES: 4-(2-hydroxyethyl)-1-piperazinethanesulfonic acid for pH 6.8 to 8.2, HEPPS: 4-(2-hydroxyethyl)-piperazin-1-propansulfonic acid for pH 7.3 to 8.7, barbital-acetate buffer, MES: 2-(N-morpholino)ethansulfonic acid for pH 5.2 to 6.7, carbonic acid-bicarbonate system for pH 6.2 to 8.6; neutral, carbonic acid-silicate buffer for pH 5.0 to 6.2; weakly acidic, acetic acid-acetate buffer for pH 3.7 to 5.7, phosphate buffer: NaH₂PO₄ +Na₂HPO₄ for pH 5.4 to 8.0, ammonia buffer NH₃+H₂O+NH₄Cl for pH 8.2 to 10.2, citric acid or citrate buffer. Particularly preferred buffer systems comprising lactic acid buffer systems, EDTA, or barbital-acetate buffer and, in the mouthwash, TRIS (tris(hydroxymethyl)-aminomethane) buffer. TRIS (Tris(hydroxymethyl)-aminomethane) is used in the mouthwash, which is a synonymous term for pre-treatment solution.

Phosphates that can be used according to the invention to produce the phosphate-containing mineralization matrices comprise phosphates, hydrogenphosphates or phosphates that can be hydrolyzed to form water-soluble phosphate ions, comprising
a) alkali phosphates, alkaline earth phosphates, dihydrogen phosphates, sodium dihydrogenphosphate, NaH₂PO₄, potassium dihydrogenphosphate, KH₂PO₄, hydrogenphosphates, dipotassium hydrogenphosphate, K₂HPO, disodium hydrogenphosphate, Na₂HPO₄, phosphate esters, monoesters, diesters, and triesters of phosphates, sodium phosphate, Na₃PO₄, potassium phosphate, K₃PO₄, calcium dihydrogenphosphate, Ca(H₂PO₄)₂, monoesters, diesters, and triesters calcium hydrogenphosphate, CaHPO₄, calcium phosphate, Ca₃(PO₄)₂ and/or
b) the calcium ions or compounds releasing calcium ions comprise calcium chloride, calcium dichloride dihydrate, calcium salt of a carboxylic acid comprising alkylcarboxylic acids, hydroxycarboxylic acid, dicarboxylic acids, fruit acids, amino acids, such as calcium lactate, calcium gluconate, calcium lacto-gluconate, calcium-alginate, calcium-L-ascorbate, compounds releasing poorly water-soluble calcium ions in delayed manner comprising calcium sulfate, calcium apatite, calcium-carbonate, calcium oxalate, calcium phosphate, calcium alginate, preferably having a particle size of less than 100 μm, preferably about 10 μm, particularly preferably of less than or equal to 5 μm, for example up to 1 μm or 50 nm or preferably mixtures of water-soluble and poorly water-soluble calcium ions or compounds releasing calcium ions. The poorly water-soluble compounds releasing calcium ions in delayed manner are added to the gel containing calcium ions that are easily soluble in water in order to improve the texture of the, in some cases, tacky gels. A total of 1 to 50% by weight compounds releasing poorly soluble calcium ions, preferably 5 to 30% by weight with respect to the total composition of the mineralization matrix, can be used.

According to an embodiment of the invention, a formulation for producing an aqueous mouthwash for pre-treating the teeth is disclosed that contains at least one calcium salt that dissolves well in water, preferably comprising calcium lactate, calcium-chloride, calcium gluconate, calcium lacto-gluconate, a hydrate of the salts or a mixture containing at least two of the salts, optionally a content of a buffer system, in particular TRIS (tris(hydroxymethyl)-aminomethane), optionally a content of masking agent or flavouring agent and common formulation excipients for producing a pre-treatment solution or together with water as pre-treatment solution prior to a pre-treatment, in which apatite is deposited on vertebrate teeth.

TRIS (Tris(hydroxymethyl)-aminomethane) is used in the mouthwash (pre-treatment solution). Also a subject matter of the invention is a formulation in the form of an aqueous mouthwash comprising water, 0.01 to 2 mol of a calcium salt that dissolves well in water, with respect to the total composition, optionally 0.01 to 0.5 mol, preferably 0.05 to 0.2 mol of a buffer system, in particular TRIS (tris(hydroxymethyl)-aminomethane), optionally masking agent or flavouring agent, and having a pH value of 5.0 to 12.0. The mouthwash or pre-treatment solution can also be used to treat single teeth.

According to another alternative embodiment, the invention discloses a formulation of an aqueous mouthwash, preferably for use in combination with any aforementioned formulation according to the invention, comprising water, 0.1 to 30% by weight of a calcium salt that dissolves well in water, in particular 5 to 20% by weight, preferably 5 to 15% by weight, with respect to the total composition, preferably comprising calcium lactate, calcium chloride, calcium gluconate, calcium lacto-gluconate, a hydrate of the salts or a mixture containing at least two of the salts, optionally a content of a buffer system, in particular Tris, optionally masking agent or flavouring agent and comprising a pH value of 5.0 to 12.0.

Also a subject matter of the invention is a method for producing a formulation that contains phosphate ions, and a formulation that can be obtained according to said method for deposition of apatite, in particular for biomimetic deposition of apatite, selected from fluorapatite, hydroxylapatite or mixtures thereof on teeth of vertebrates, comprising
(1) producing at least one partially elastic shaped body, in particular shaped body A, comprising at least one mineralization matrix containing at least one gel containing water-soluble phosphates or phosphates that can be hydrolyzed to form water-soluble phosphate ions, whereby the shaped body comprises, at least in part, in at least one plane, a reduced solubility with respect to aqueous media as compared to the mineralization matrix, in particular on at least one outer surface, particularly preferably on multiple planes forming an envelope, whereby the plane acts, in particular as H₂O, peptide, and ion-permeable membrane, and producing the shaped body through preparing,
a) for producing at least one mineralization matrix containing the gel, also called phosphate component A, in a first step, a mixture of
(i) 0.05 to 4 mol/l, 0.5 to 1.5 mol/l water-soluble phosphates or phosphates that can be hydrolyzed to form water-soluble phosphate ions;
(ii) a corresponding amount of water or of a mixture of water and an organic solvent;
(iii) optionally at least one carboxylic acid and/or a buffer system, in particular for adjusting the pH value to 2 to 8, preferably 3.5 to 8, more preferably 3.5 to 6, particularly preferably about 5.5 plus/minus 0.5;
(iv) 0 to 6,000 ppm by weight water-soluble fluoride or compound releasing fluorides, in particular 1 to 4,000 ppm by weight, more preferably 500 to 2,500 ppm by weight, particularly preferably about 2,000 ppm by weight plus/minus 500 ppm by weight, is prepared, and using, in a further step, the mixture produced in a)
b) together with gelatine and optionally glycerol, while heating, to produce the gel;
c) forming, such that the mineralization matrix is formed, optionally solidification, and a plane, in particular the envelope, of the at least one mineralization matrix arranged on the outer surface is formed in a subsequent step d), while the shaped body is being formed.

The plane or envelope can be produced through cross-linking or through application of a coating. For cross-linking the at least one plane of the at least one mineralization matrix that is arranged on the outer surface, in particular in the form of a flat element or at least partial negative image of a jaw, the plane is exposed to a mixture, preferably an aqueous solution, containing a di- or polyfunctional cross-linker in a further step.

The phosphate solution for producing phosphate component A contains, inter alia, a water-soluble phosphate salt. For example alkali salts, such as sodium or potassium phosphates, hydrogen or dihydrogen phosphates are well-suited. The listing is inclusive, but not exclusive. The concentration of the phosphate salts in the solution is between 0.05 and 4 mol/l Gel, preferably 0.5 to 1.5 mol/l, particularly preferably about 1 mol/l plus/minus 0.5 mol/l. In addition, the phosphate solution contains a water-soluble fluoride salt, e.g. an alkali salt, or tin fluoride or Olafluor. The listing is inclusive, but not exclusive. The concentration of the fluoride in the solution is between 0 and 6,000 ppm by weight, preferably 200 to 4,000 ppm by weight, particularly preferably 2,500 to 4,000 ppm by weight or about 3,000 ppm by weight plus/minus 500 ppm by weight. The phosphate solution can be used as a cross-linker solution, e.g. upon adding the cross-linker, for example glutardialdehyde.

The pH value of the phosphate solution is between 2.0 and 8.0, preferably between 3.5 and 5,5, and is adjusted using a suitable buffer system. Carboxylic acids, such as ascorbic acid, pyruvic acid, tartaric acid, acetic acid, lactic acid or malic acid, but all other buffer systems just as well, are particularly well-suited. The concentration of the buffer is between 0.25 and 4.0 mol/l, preferably between 0.5 and 1.5 mol/l.

The solution is used to produce a gelatine-glycerol gel. The amount of gelatine preferably is 25 to 40% by weight and the amount of glycerol is 5 to 20% by weight with respect to the total composition of aqueous gel. In order to mix the components homogeneously, the preparation is heated to 40 to 90° C., preferably to 50 to 70° C.

The thickness of phosphate component A in this context is 50 to 3,000 μm, preferably 200 to 2,000 μm, particularly preferably 300 to 1,500 μm.

Also a subject matter of the invention is a method for producing a formulation containing calcium ions and a formulation that can be obtained according to said method for deposition of apatite, in particular for biomimetic deposition of apatite, selected from fluorapatite, hydroxylapatite or mixtures thereof on teeth of vertebrates, comprising
(1) producing at least one partially elastic shaped body, in particular shaped body B, comprising at least one mineralization matrix containing at least one gel containing calcium ions or compounds releasing calcium ions, whereby the shaped body comprises, at least in part, in at least one plane, a reduced solubility with respect to aqueous media as compared to the mineralization matrix, in particular on at least one outer surface, particularly preferably on multiple planes forming an envelope, whereby the plane acts, in particular, as membrane, and producing the shaped body through preparing,
a) for producing at least one mineralization matrix containing the gel, also called calcium component B, in a first step, a mixture of
(i) 0.1 to 2 mol/l calcium ions or compounds releasing calcium ions;
(ii) a corresponding amount of water or of a mixture of water and an organic solvent;
(iii) optionally, at least one carboxylic acid and/or a buffer system, in particular for adjusting the pH value to 3.5 to 14, preferably 4.0 to 6.0 or 6.0 to 11.0, preferably 4.0, particularly preferably about 4.0 plus/minus 0.5; is being prepared, and using, in a further step, the mixture produced in a)
b) together with gelatine and optionally glycerol, while heating, to produce the gel;
c) forming, such that the mineralization matrix is formed, optionally solidification, and a plane, in particular the envelope, arranged at the outer surface of the at least one mineralization matrix is formed in a subsequent step d), while the shaped body is being formed. The plane or envelope can be produced through the aforementioned cross-linking or through application of a coating. A certain degree of porosity is crucial in the production of the plane or envelope in order to enable the deposition of the bio-composites.

For producing the aforementioned formulations, in b), 5 to 50% by weight gelatine with respect to the total composition of the gel and 0 to 30% by weight glycerol with respect to the total composition of the gel are added each independently in a further step, preferably 25 to 40% by weight gelatine and 5 to 20% by weight glycerol are added to produce the formulation containing the mineralization matrix containing water-soluble phosphates or phosphates that can be hydrolyzed to form water-soluble phosphate ions, and 20 to 40% by weight gelatine and 15 to 25% by weight glycerol are added to produce the formulation containing the mineralization matrix containing calcium ions or compounds releasing calcium ions.

The gelatine-containing formulations in step b) are preferably heated to 40 to 90° C. in order to homogeneously mix the components, preferably, the temperature range is 50 to 70 ° C.

The solution for producing calcium component B contains a water-soluble calcium salt, e.g. calcium chloride or calcium lactate or calcium gluconate or calcium lacto-gluconate. The listing is inclusive, but not exclusive. The concentration is between 0.1 and 2.0 mol/l, preferably between 0.5 and 1.5 mol/l. The pH value between 4.0 and 14.0, preferably between 6.0 and 11.0, is adjusted using a suitable buffer system. Carboxylic acids, such as ascorbic acid, pyruvic acid, tartaric acid, acetic acid, lactic acid or malic acid, but all other buffer systems with a suitable pKs just as well, are particularly well-suited. The concentration of the buffer is between 0.1 and 3.0 mol/l, preferably between 0.25 and 1.0 mol/l. The solution is used to produce a gelatine-glycerol gel. The amount of gelatine preferably is 20 to 40 weigth-% by weight with respect to the total composition of aqueous gel and the amount of glycerol is 15 to 25% by weight. Since the calcium-gelatine solution is very tacky even after gelling and thus is unpleasant to handle, a poorly soluble calcium salt is added to improve the texture. Calcium sulfate, calcium apatite, calcium carbonate, calcium oxalate are particularly well-suited. The listing is inclusive, but not exclusive. In order to obtain a particularly homogeneous paste, it is advantageous for the particle sizes to be less than 10 μm. It is preferred to use particles with particle sizes of less than 1 μm. The amount of the poorly soluble calcium salt added preferably is 1 to 50%, very preferably 5 to 30 weight-%. In order to mix the components homogeneously, the preparation is heated to 40-90° C., preferably to 50 to 70° C.

The thickness of calcium component A in this context is 10 to 3,000 μm, preferably 100 to 1,500 μm, particularly preferably 300 to 1,500 μm, even more preferably 500 to 1,500 μm.

In order to produce the shaped bodies of the formulations, the not-yet-solidified gels are formed and then solidified. Therefore, a method, in which the gel produced in further step d) is being formed and can be solidified also is a subject matter of the invention. Flat elements or a custom three-dimensional shape, preferably in the shape of a negative image of a jaw, are preferred.

The gels for formation of the mineralization matrix can be formed by filling them in moulds, extrusion, forming into flat elements by painting, distribution, streaking out, pressing through appropriately shaped nozzles, followed by a solidification step. Extrusion of the gels into any shape is preferably possible with pasty gels. The solidification usually proceeds as early as during the cooling process. In general, the gel can be converted into any shape and solidified and optionally be provided with an essentially complete or partial envelope in order to prevent it from dissolving in the oral environment or under physiological conditions.

The mineralization matrices are chemically cross-linked in order to prevent the mineralization matrices from dissolving in the oral environment. In this context, the degree of cross-linking has to be selected appropriately such that the polypeptides are still available for formation of the composite, while sufficient stability in the oral environment and/or under physiological conditions is guaranteed. It is sensible for the gel to already have the desired shape at the time of cross-linking. This can either be strips of certain dimensions or three-dimensional bodies in the form of a negative image of the jaw. The gel moulds can be taken out after cross-linking, and optionally after subsequent drying to a water content between 8 and 60% by weight, preferably 30 to 55% by weight, while obtaining the two-dimensional or three-dimensional moulds.

The drying further stabilises the gel moulds. The degree of cross-linking can be adjusted by means of the type and concentration of cross-linker, the pH value of the cross-linker solution, and the time of action. All di- or polyfunctional compounds capable of reacting with multiple side groups of gelatine, such as dialdehydes, polyepoxides or polyisocyanates, are well-suited for cross-linking. It is preferable to use glutardialdehyde which affords the further advantage of having a disinfecting effect and being volatile.

The cross-linking of at least one plane of the at least one mineralization matrix that is arranged on the outer surface, preferably in the form of a flat element or at least partial negative image of a jaw, proceeds in a further step by contacting the mineralization matrix to a mixture, preferably an aqueous solution, containing a di- or polyfunctional cross-linker comprising dialdehydes, polyepoxides, polyisocyanates. In this step, the essentially water-insoluble cross-linking to the gel is formed. The contacting affords at least partial chemical cross-linking in at least one plane of the flat element that is arranged on the outer surface while obtaining the at least one shaped body comprising at least one plane comprising a reduced solubility with respect to aqueous media as compared to the mineralization matrix. According to an alternative embodiment, all planes of the shaped body, such as flat element, that are arranged on the outer surfaces are cross-linked in order to produce a cross-linked envelope of the at least one mineralization matrix while obtaining the at least one shaped body.

Another subject matter of the invention are two cross-linker solutions, in particular for use in the production, each independently, of a formulation in the form of a shaped body, comprising a cross-linker solution (a) comprising a phosphate mixture produced by mixing
(i) 0.05 to 4 mol/l, 0.5 to 1.5 mol/l water-soluble phosphates or phosphates that can be hydrolysed to form water-soluble phosphate ions,
(ii) a corresponding amount of water or of a mixture of water and an organic solvent,
(iii), optionally, at least one carboxylic acid and/or a buffer system, (iv) 0 to 6,000 ppm by weight water-soluble fluoride or a compound releasing fluoride, and/or a cross-linker solution, (b) comprising a calcium mixture produced by mixing
(i) 0.1 to 2 mol/l calcium ions or compounds releasing calcium ions,
(ii) a corresponding amount of water or of a mixture of water and an organic solvent,
(iii), optionally, at least one carboxylic acid and/or a buffer system, and mixing (a) and/or (b) with a defined amount of a solution containing a di- or polyfunctional cross-linker comprising dialdehydes, polyepoxides, polyisocyanates, whereby the cross-linker, linker, in particular, is glutardialdehyde. The phosphate solution can be used as a cross-linker solution, e.g. upon adding the cross-linker, for example glutardialdehyde.

Likewise, the calcium solution can be used as a cross-linker solution, e.g. upon adding the cross-linker, for example glutardialdehyde. For this purpose, the cross-linker is present in a cross-linker solution and is contacted to the gel thus formed. Solutions containing 0.005 to 90% by weight cross-linker in solvent with respect to the total composition, in particular in water or water-containing solvent are preferred, whereby 0.005 to 5% by weight are preferred, 0.1 to 4% by weight are particularly preferred, and about 0.1 to 1% by weight are advantageous. It is preferred to use an aqueous glutardialdehyde solution as cross-linker solution. The cross-linker solution is preferably prepared by adding the cross-linker to the phosphate solution. The preferred time of action is 1 to 200 seconds, particularly preferably 10 to 60 seconds (s). Preferably, the treatment takes approx. 20 seconds. The preferred pH value of the cross-linker solution is between 4.0 and 12.0. Final rinsing with phosphate and/or calcium solution is feasible. Also a subject matter of the invention is a cross-linker solution comprising a phosphate solution or a calcium solution and a content of cross-linker.

The production of shaped bodies containing one matrix each proceeds as follows: at least one mineralization matrix, in particular in the form of a flat element or at least partial negative image of the jaw, for producing a shaped body containing phosphates and fluorides is cross-linked in at least one plane that is arranged on the outer surface, or as envelope.

The production of a mineralization matrix for producing a shaped body containing calcium ions proceeds analogously. Shaped bodies containing both mineralization matrices can be produced as follows: arranging (i) at least one mineralization matrix containing a gel in the form of a flat element comprising phosphates and fluorides, (ii) optionally separated by a membrane (layer), and (iii) a second mineralization matrix arranged on it containing a gel in the form of a flat element comprising calcium ions; external cross-linking or coating of the shaped body containing flat elements (i), (ii), and, optionally, (iii), in order to provide the envelope. Alternatively, one or both mineralization matrices can be coated with a coating with membrane-forming, polymeric coatings, in particular containing acrylates such as EUDRAGIT polymers or polyvinylalcohol (PVA), for example from Merck Millipore.

Also a subject matter of the invention is a kit comprising a partially elastic shaped body A, in particular a formulation of a partially elastic shaped body A, and a partially elastic shaped body B, in particular a formulation of a partially elastic shaped body B, which each, independently, are present in the form of a three-dimensional body, in particular as a flat element or at least partial negative image of a jaw, whereby (a) partially elastic shaped body A comprises (a1)) at least one mineralization matrix comprising at least one gel, (a2) at least one water-soluble phosphate or phosphates that can be hydrolyzed to form water-soluble phosphate ions, and (a3), optionally, at least one carboxylic acid and/or a buffer system, (a4), optionally, water-soluble fluorides or a compound releasing fluorides, (a5), optionally, a content of water or of a mixture of water and an organic solvent;
(b) partially elastic shaped body B comprises (b1) at least one mineralization matrix comprising at least one gel, (b2) water-soluble calcium ions or compounds releasing calcium ions; and
(b3), optionally, at least one carboxylic acid and/or a buffer system, (b4), optionally, water or a mixture of water and an organic solvent, or at least one partially elastic shaped body C, whereby (c) partially elastic shaped body C comprises (c1) at least one mineralization matrix comprising at least one gel, containing (c1.1) at least one water-soluble phosphate or phosphates that can be hydrolyzed to form water-soluble phosphate ions, and (c1.2), optionally, at least one carboxylic acid and/or a buffer system, (c1.3), optionally, water-soluble fluorides or a compound releasing fluorides, (c1.4), optionally, a content of water or of a mixture of water and an organic solvent, (c2), optionally, a membrane (layer), (c3) at least one mineralization matrix comprising at least one gel, containing (c3.1) water-soluble calcium ions or compounds releasing calcium ions, and (c3.2), optionally, at least one carboxylic acid and/or a buffer system, (c3.3), optionally, water or a mixture of water and an organic solvent, whereby the layer structure of partially elastic shaped body C is c1 and c3 or c1, c2, and c3. Alternatively, shaped body C can just as well comprise two mineralization matrices c1 and c1′ containing different phosphate contents in order to provide a concentration gradient in the phosphate component. Accordingly, a shaped body C can just as well comprise two mineralization matrices c3 and c3′ differing in concentration.

The water content of the mineralization matrix remains essentially unchanged. Whereby the mineralization matrices also are present, preferably each independently, in the form of a flat element or at least partial negative image of a jaw.

Moreover, a kit, in which the respective shaped body comprises, each independently, at least in part, in at least one plane arranged on the outer surface or an outer envelope, whereby the plane or envelope comprises a reduced solubility with respect to aqueous media as compared to the mineralization matrix, whereby the plane or envelope acts as membrane. And a kit comprising (I) a shaped body in the form of a flat element having at least two mineralization matrices, optionally separated by a membrane, each independently in the form of a flat element containing the gel at a layer thickness of 50 to 6,000 μm, or (II) two separate shaped bodies each independently in the form of a flat element each having at least one mineralization matrix in the form of a flat element containing the gel, whereby each shaped body independently has a layer thickness of 10 to 3,000 μm, whereby the first shaped body comprising water-soluble phosphates or phosphates that can be hydrolysed to form water-soluble phosphate ions has a layer thickness of 50 to 3,000 μm, and the second shaped body comprising calcium ions or compounds releasing calcium ions has a layer thickness of 10 to 3,000 μm. According to the alternative, i.e. the shaped body being a negative image of the jaw, the shaped body preferably comprises inner layers of the at least one or two mineralization matrix/matrices that adapt to the teeth. The layers can be applied by immersing, painting or other measures known to a person skilled in the art and can be cross-linked subsequently. The negative image of the jaw can therefore comprise a support and the mineralization matrices or can be produced directly from the mineralization matrices, for example the calcium component is the support and the phosphate component is introduced, as inner layer, in the form of a non-planar flat element.

Moreover, the kit preferably comprises a formulation in the form (a) of an aqueous pre-treatment solution, which is synonymous to mouthwash, comprising water, 0.1 to 30% by weight of a calcium salt that dissolves well in water, in particular 5 to 15% by weight with respect to the total composition, preferably calcium lactate, calcium-chloride, calcium gluconate, calcium lacto-gluconate, a hydrate of the salts or a mixture containing two of the salts, optionally a content of a buffer system, optionally masking agent or flavouring agent, and comprising a pH value of 5.0 to 12.0, or the formulation in the form b) comprises at least one calcium salt that dissolves well in water, preferably comprising calcium lactate, calcium chloride, calcium gluconate, calcium lacto-gluconate, a hydrate of the salts or a mixture containing two of the salts, optionally a content of a buffer system, optionally a content of masking agent or flavouring agent, and common formulation excipients, such as releasing agents, HPMC, etc., in particular in the form of a water-soluble granulated material, water-soluble pellet, tablets, as sachet, powder and/or granulated material in a sachet or soluble capsules. Also a subject matter of the invention is the use of a single formulation of the form of b) in combination with a kit comprising the formulations according to the invention. The afore-mentioned mouthwash is a pre-treatment solution for pre-treating single or all teeth or a formulation from which a corresponding solution can be produced by adding water.

The composition of the kit is described in the following. The mouthwash or pre-treatment solution is composed of 0.1% by weight to 30% by weight of a calcium salt that dissolves well in water, preferably 5% by weight to 15% by weight calcium chloride or calcium lactate or calcium gluconate or calcium lacto-gluconate or other sufficiently soluble calcium salts. The solution is adjusted to a pH value between 5.0 and 12.0, preferably 8.0 to 10.0, using a suitable buffer. To improve the taste, flavouring or complexing of bad tasting compounds is feasible as long as this does not have a detrimental effect on the deposition of apatite. Suitable as buffers are all buffers showing good buffering capacity in said pH range, e.g. EDTA, Tris, HEPES or barbital-acetate buffer, but other buffer systems as well, with Tris being preferred.

Moreover, it is preferred that the formulations or mouthwashes comprise at least one buffer system, preferably a buffer substance from the group of primary alkali citrates, secondary alkali citrates and/or a salt of carboxylic acids, in particular having 1 to 20 C atoms, preferably a salt of at least one fruit acid, a salt of an alpha-hydroxy acid and/or a salt of a fatty acid, in particular having 1 to 20 C atoms. Particularly preferred salts of the aforementioned acids are the alkali, alkaline earth and/or zinc salts of citric acid, malic acid, tartaric acid and/or lactic acid or Tris. Particularly preferred salts comprise the cations of sodium, potassium, magnesium and/or zinc of the aforementioned carboxylates.

Also a subject matter of the invention is the use of the formulations or of a kit for depositing crystalline fluorapatite, fluorapatite crystals, needle-shaped fluorapatite, enamel-like coatings made of apatite on dental hard substance, apatite crystals, apatite on surfaces, fluorapatite, hydroxylapatite, fluoro- and hydroxylapatite, on teeth, on enamel, for morphological modification of tooth surfaces, for sealing lumens or channel structures in teeth, in lumens, on dentine, for sealing open dentine tubuli, for depositing apatite on bone, on a bone matrix, a formulation and/or a shaped body in the form of a negative mould for depositing apatite in lumens or cavities of a tooth, in particular treated lumens of a tooth. Also a subject matter of the invention is the use of a formulation or of a kit for depositing apatite, in particular fluorapatite, on surfaces at a layer thickness of more than or equal to 1 μm, preferably more than or equal to 2 μm, within 24 hours, within two times 12 hours, preferably within 16 hours, in particular at body temperature of 37° C. in the oral environment, at 37° C. and 95% humidity. Preferably, the formulation according to the invention needs to be applied just once or twice for 4 to 12 hours each in order to obtain an essentially surface-covering, preferably crystalline apatite deposit having a layer thickness of more than or equal to 2 μm, better of more than or equal to 4 to more than or equal to 5 μm.

Use of a formulation containing at least one calcium salt that dissolves well in water, as disclosed above, optionally a content of a buffer system, optionally a content of a masking agent or flavouring agent as well as common excipients for formulation for producing a pre-treatment solution, which is synonymous to mouthwash, or, in combination with water, as pre-treatment solution prior to a treatment, in which apatite is deposited on vertebrate teeth.

Use of a formulation containing water-soluble phosphate or phosphates that can be hydrolysed to form water-soluble phosphate ions, water-soluble fluorides or a compound releasing fluorides, and water-soluble calcium ions or compounds releasing calcium ions for a remineralising treatment of teeth by means of once or twice daily oral application of the formulation of the partially elastic shaped body comprising at least a partial envelope, preferably an essentially complete envelope, before going to bed and use of the formulation over-night or during the day to form, in particular, crystalline apatite deposits, fluorapatite, in particular having a layer thickness after single application of more than or equal to 2 μm, preferably of more than or equal to 4 μm. In this context, the following shaped bodies having a very low layer thickness are already sufficient for said use: (I) shaped body in the form of a flat element or negative image of the jaw having flat elements, which are arranged on the inside, for layers, having at least two mineralization matrices, optionally separated by a membrane, containing the gel at a layer thickness of 50 to 6,000 μm, preferably 50 to 750 μm, particularly preferably 50 to 600 μm, or (II) two separate shaped bodies each independently having at least one mineralization matrix containing gel, whereby each shaped body independently has a layer thickness of 10 to 3,000 μm, whereby the first shaped body comprising water-soluble or phosphates that can be hydrolysed to form water-soluble phosphate ions has a layer thickness of 50 to 3,000 μm and/or the second shaped body comprising calcium ions or compounds releasing calcium ions has a layer thickness of 10 to 3,000 μm.

Preferably, the formulations according to the invention can be used for treatment of sensitive teeth, sensitive dental necks, acid-eroded teeth, cracked teeth, surface-abraded teeth, exposed dental necks, bleached teeth, teeth after treatment of carious tooth regions once (once-a-day) or twice (for example, one-day-mineralization) in order to form an apatite layer, which preferably is homogeneous and which essentially is crystalline, of 2 to more than or equal to 5 μm in thickness on the treated surfaces. As a matter of principle, the formulation can be used more frequently according to need, for example according to defined intervals.

FIGS. 9A and 9B disclose general embodiments of the shaped bodies according to the invention.

FIG. 9A shows two shaped bodies 0, whereby the mineralization matrix 2 comprises the calcium component and the mineralization matrix 1 comprises the phosphate component.

The envelope (cross-linking, coating) is indicated by 4 and the optional membrane (layer) by 5.

FIG. 9A shows two shaped bodies having one mineralization matrix each and FIG. 9B shows a shaped body having two mineralization matrices in an envelope. The mineralization matrices can just as well each contain calcium or phosphate at different concentrations.

The invention is illustrated in more detail based on the following examples and figures without limiting the invention to the examples given.

### Example 1 REFERENCE EXAMPLE

For component A containing phosphate ions, a solution containing 29.5 g NaH₂PO₄, 33 g Olaflur, and 27.0 g lactic acid was produced. The pH value was adjusted to 5.4 with 5 N sodium hydroxide solution and the solution was topped up to 250 ml with de-ionised water. A total of 24 ml of the solution and 6 g glycerol and 10 g of 300 Bloom pork rind gelatine were processed while heating to form a viscous solution. A small amount of liquid was placed in a template with a wall thickness of 500 μm and exposed to 2 bar of pressure. After solidification, the strips were removed from the template and cut into 1×1 cm squares.

For component B containing calcium ions, a calcium chloride solution containing 29.4 g calcium chloride dihydrate and 6.3 g lactic acid was prepared. The pH value was adjusted to 4.0with 5 N sodium hydroxide solution. The solution was topped up to 200 ml with de-ionised water. In order to produce the gel, 21.6 g of the solution and 8.24 g glycerol and 8 g calcium sulfate and 13.6 g 300 Bloom gelatine were mixed and heated. The liquid gel was then spread with a squeegee to a thickness of 1 mm or pressed in a template with a wall thickness of 1 mm. After solidification, the strips were cut into 1×1 cm squares. For the pre-treatment solution (mouthwash), a 0.1 mol Tris buffer was added to a 1 molar calcium chloride solution and the pH was adjusted to 9.0.

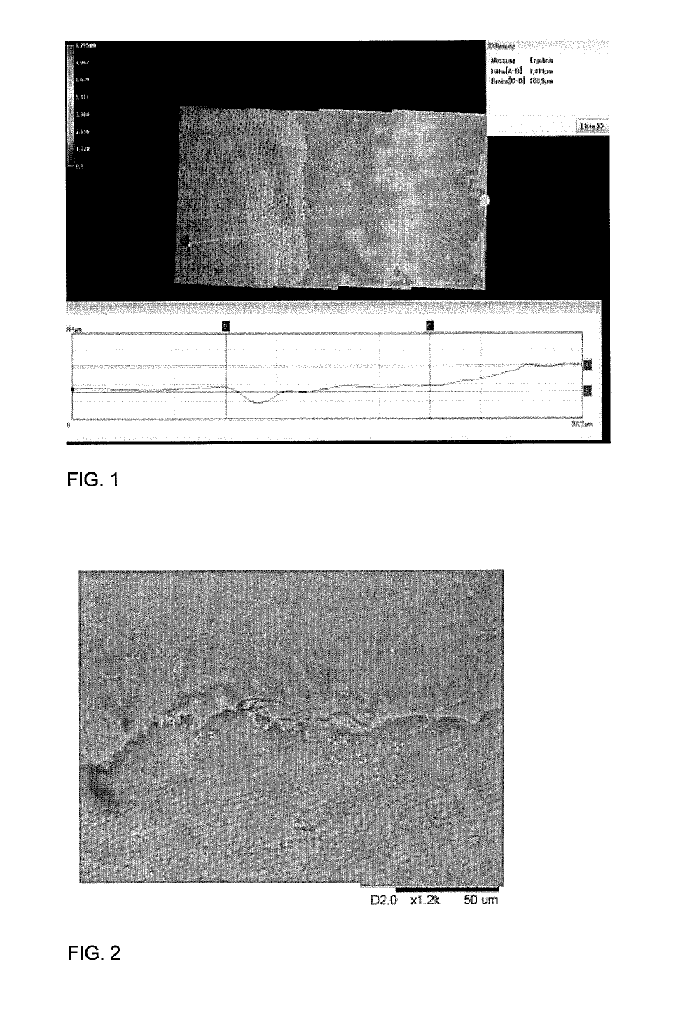

For assessment of the mineralization activity, 6 tooth discs each were etched for 10 s with 1 M HCl, rinsed with the pre-treatment solution, and covered with one piece of phosphate gel and one piece of calcium gel each. In order to make the morphological change of the tooth surface more obvious, one half of a disc was taped over first such that only half of the disc can remineralize. The samples were stored in an air-conditioned cabinet at 37° C. and 95% humidity and cleaned after 8 to 16 hours with lukewarm water and a soft toothbrush. After just one treatment, most of the tooth surface is coated by a firmly adhering layer. FIG. 1 shows the typical surface morphology of the coating at the boundary between coated and uncoated sample after one treatment. The layer can be up to 2.5 μm in thickness. The gels were spread over the samples after 8 to 16 hours. This disadvantage is not acceptable for the user for an application of the matrices in the mouth.

FIG. 1: Typical layers after one application of the mineralization kit 1: Light microscopy image in false colour, 3D microscope made by Keyence, typical layer after one application of the mineralization kit. The colours represent the different heights. The thickness of the layer can be determined from the line scan. Left: untreated dentine (blue) right: treated dentine (green-red). The channel structure disappears under a dense layer (3D microscope, Keyence), which can be up to 2.5 μm in thickness. FIG. 2: SEM image of the boundary between coated enamel and uncoated enamel).

### EXAMPLE 2

The components were produced as described in example 1 except that the pH value of the phosphate component was adjusted to 5.6 and the pH value of the calcium component was adjusted to 4.3. A cross-linker solution for the phosphate component was prepared. For this purpose, 1.5 g of 25% by weight glutardialdehyde solution were topped up to 100 g with the phosphate (P) solution prepared before. For cross-linking, the gel plates were cut into 1 cm2-sized squares and placed on a film. Subsequently, they were dipped into the cross-linker solution for 20 s. Subsequently, they were rinsed with the P solution containing no cross-linker component, and blown dry. A cross-linker solution for the Ca component was prepared. For this purpose, 2 g of 25% by weight glutardialdehyde solution were topped up to 100 g with the Ca solution prepared before. For cross-linking, the strips were cut into 1 cm2-sized squares, placed on the film, and exposed to the cross-linker solution for 40 s. Subsequently, they were rinsed with the Ca solution containing no cross-linker component, and blown dry.

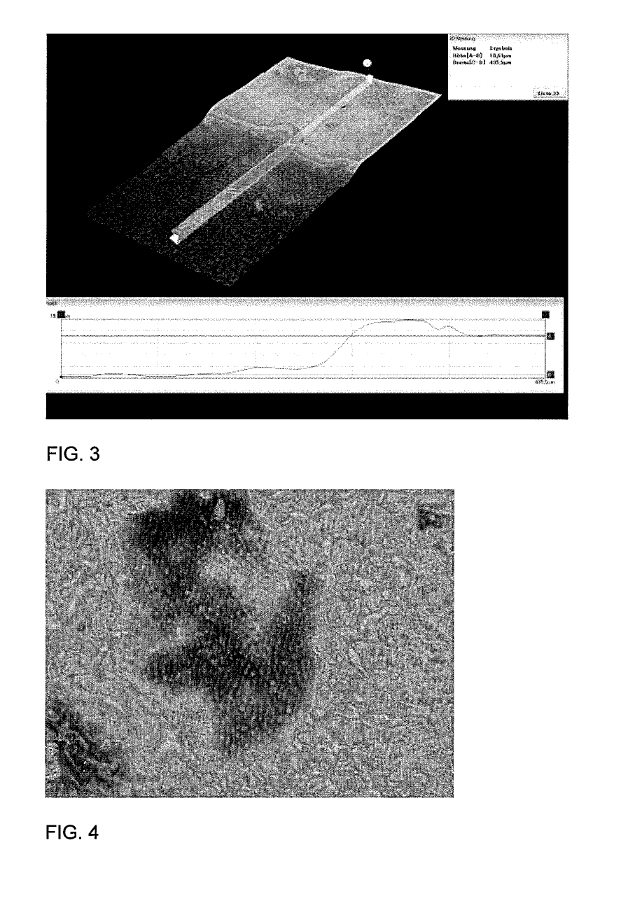

For assessment of the mineralization activity, 6 tooth discs each were rinsed with the pre-treatment solution and covered with one piece of phosphate gel and one piece of calcium gel each. In order to make the morphological change of the tooth surface more obvious, one half of a disc was taped over first such that only half of the disc can remineralise. The samples were stored in an air-conditioned cabinet at 37° C. and 95% humidity and cleaned after 12 hours with lukewarm water and a soft toothbrush. After just one treatment, part of the tooth surface is completely covered by a firmly adhering layer. FIG. 4 shows the typical surface morphology of the coating at the boundary between coated and uncoated sample after one treatment. The layer can be up to 7 μm to 10 μm thick in individual places, especially at the boundary. On average, the thickness of the layer is 2 to 3 μm. In order to render the regions of successful coating versus exposed dentine more obvious to the naked eye, the samples were cleaned and then soaked in 0.1% by weight rhodamine solution and rinsed briefly. The porous regions of dentine stain pink-red, whereas the areas with growth no longer absorb colour due to their density. As a result, regions that are not successfully coated can be recognized easily (see FIGS. 3 and 4).

FIG. 3: Typical layer after one application of the mineralization kit and staining with 0.1% rhodamine solution. Left: untreated dentine is dark (or red) Right: bright treated dentine (white), the apatite layer is up to 7 μm to 10 μm in thickness, in particular at the boundary. The channel structure disappears under a dense layer. FIG. 4 Partially unsuccessfully coated regions can be made obvious by staining (FIGS. 1, 3, and 4: 3D microscope, Keyence).

### EXAMPLE 3

The components were produced as described in example 1. However, the pH value of the Ca solution was adjusted to 4.01. A cross-linker solution for the P component was prepared. For this purpose, 2 g of 25% by weight glutardialdehyde solution were topped up to 100 g with the P solution prepared before. For cross-linking, the strips were cut into 2 cm² -sized squares and swirled in the cross-linker solution for 40 s. Subsequently, they were rinsed with the P solution containing no cross-linker component, and blown dry. A cross-linker solution for the Ca component was prepared. For this purpose, 2 g of 25% by weight glutardialdehyde solution were topped up to 100 g with the Ca solution prepared before. For cross-linking, the strips were cut into 2 cm²-sized squares and swirled in the cross-linker solution for 40 s. Subsequently, they were rinsed with the Ca solution containing no cross-linker component, and blown dry.

For assessment of the mineralization activity, the roots of 2 whole human frontal teeth were embedded in resin at a distance of maximally 1 mm from each other, which affords a simulated intendental space. A customized deep-drawing splint leaving 1.5 mm of space for the gel component was produced for said row of teeth. The three-dimensional gel moulds were shortened somewhat, if needed, and covered with one piece of phosphate gel and one piece of calcium gel each. In order to make the morphological change of the tooth surface more obvious, one half of a disc was taped over first such that only half of the disc can remineralize. The samples were stored in an air-conditioned cabinet at 37° C. and 95% humidity and cleaned after 12 to 16 hours with lukewarm water and a soft toothbrush. After just one treatment, part of the tooth surface is completely covered by a firmly adhering layer.

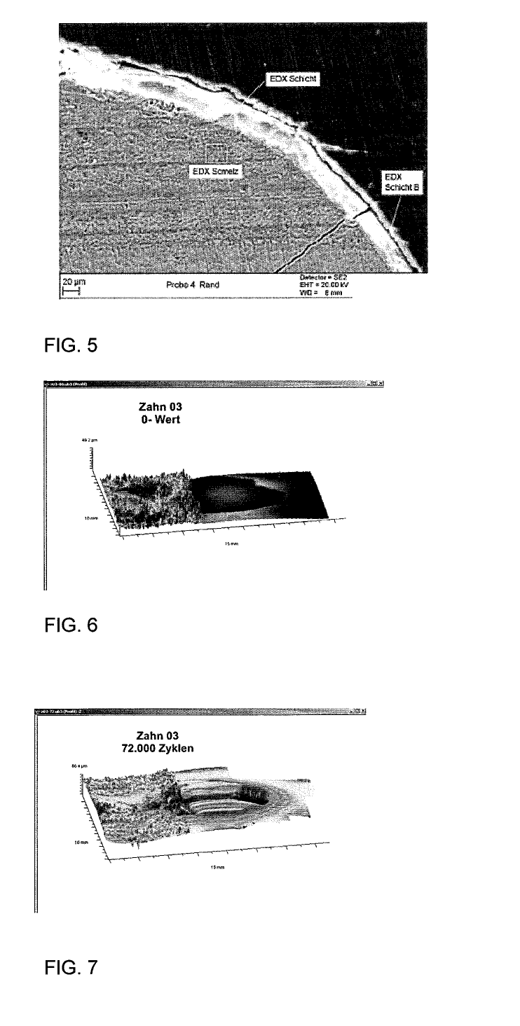

For identification of the layer by means of EDX analysis, the teeth were sawed in transverse direction such that the layer of growth can be recognized in the cross-section. FIG. 5 shows a detail of the upper edge of the tooth including the fluoride-rich layer. The analysis evidencing fluoride, calcium, and phosphate was interpreted as proof of the formation of a fluoride-containing calcium phosphate. In general, no fluoride is detected in natural enamel by means of EDX. The thickness of the layer of far more than 20 μm as measured here cannot be correlated to the actual layer thickness of approx. 5 μm, since the preparation involves the layer being sawed in oblique direction making it appear thicker in the cross-section than it actually is. FIG. 5: SEM image of a human tooth (enamel) sawed transverse to the labial surface after 2 treatments with a mineralization kit. Whereas an analysis of the enamel showed only calcium and phosphate as the main components, fluoride can be seen in the mineralized layer in addition to calcium and phosphate, which is interpreted to be indicative of artificial enamel thus generated. The layer gets increasingly thinner in the interdental space. It was possible to detect the mineralized layer by EDX analysis and to differentiate it from natural enamel not only by the changed morphology, but also by its increased fluoride content. In line with the composition of apatite, the main elements detected were Ca, P, O, and F. The percentage values are semi-quantitative, since the EDX unit was not calibrated using a fluorapatite standard. The analysis detected approx. 5.55% fluorine and approx. 55.52% oxygen, 0.57% sodium, 12.39 phosphorus, 0.24% chlorine, and 15.34% calcium.

### EXAMPLE 4a

For production of the P solution of the P component, 5.9 g Na₂HPO₄, 9.1 g lactic acid, 6.6 g Olaflur, and 0.6 g 5 M NaOH were topped up to 50 ml with de-ionized water. The gel was produced as described in example 1. The calcium solution for the Ca component was produced by dissolving 14.7 g CaCl2, 3.15 g lactic acid, 10 g 5 M sodium hydroxide solution in de-ionized water to produce a total of 100 ml of the solution. The gel was produced as described in example 1. The same applies to the pre-treatment solution. For assessment of the mineralization activity, 6 tooth discs each are etched for 10 s with 1 M HCl, rinsed with the pre-treatment solution, and then covered with one piece of phosphate gel and one piece of calcium gel each. In order to make the morphological change of the tooth surface more obvious, one half of a disc was taped over first such that only half of the disc can remineralize. The samples were stored in an air-conditioned cabinet at 37° C. and 95% humidity and cleaned after 8 to 12 hours with lukewarm water and a soft toothbrush. After just one treatment, most of the tooth surface is coated by a firmly adhering layer. In order to render the regions of successful coating versus exposed dentine more obvious to the naked eye, the samples were cleaned and then soaked in 0.1% rhodamine solution and rinsed briefly. A colorimeter was used to determine the colour difference delta E between the coated and the uncoated side. The average value is 52 (STAB 12).

### EXAMPLE 4b

The production of the kit components corresponds to example 4a, except for the P gel being treated for 30 s with an 0.375% GDA solution produced by mixing the P solution with the appropriate amount of GDA. The gel strips were then only dabbed to dry them. The Ca gels were treated with a 0.25% GDA solution for 30s and then dabbed dry. The mineralization activity was assessed as in example 4a. The average value of delta E was 63 (STAB 5, error specification).

### EXAMPLE 5 (REFERENCE EXAMPLE)

The gels were produced as in example 1. For assessment of the mineralization activity, 6 tooth discs each were covered with one piece of phosphate gel and one piece of calcium gel each. In order to make the morphological change of the tooth surface more obvious, one half of a disc was taped over first such that only half of the disc can remineralize. The samples were kept in an air-conditioned cabinet at 37° C. and 95% humidity, and washed and subjected to another gel treatment daily. After three treatments, the tooth surface was basically completely covered by a firmly adhering layer.

The enamel-like stability can be shown by means of toothbrush abrasion tests. After 72,000 homogeneous brush strokes (150 g load) on a tooth sample treated on half of a side, it was evident that the unprotected dentine was abraded markedly more strongly than the protected side, which was barely abraded, much like natural enamel. FIGS. 6 and 7: 3× tooth discs treated on half of a side before (left) and after toothbrush abrasion. It is clearly evident that treatment with the mineralization kit protects dentine from abrasion, since the untreated dentine is abraded strongly, as is evident from a 3D image of biomimetically treated teeth, on which fluorapatite was deposited, versus untreated teeth, whose two tooth surfaces were subsequently abraded by means of a toothbrush. The untreated teeth were abraded strongly. The gels (with no chemically cross-linked plane or envelope) were spread after each treatment. This disadvantage is not acceptable for the user for an application of the matrix in the mouth.

### EXAMPLE 6

The gels were produced as in example 4b. However, the cross-linking proceeded for 2×20 s from both sides with both gels. In this context, the GDA concentration of the Ca cross-linker solution was 0.5%. The mineralization activity was assessed as in the previous examples. A largely homogeneous layer of small needle-like crystals was seen in the electron microscope.

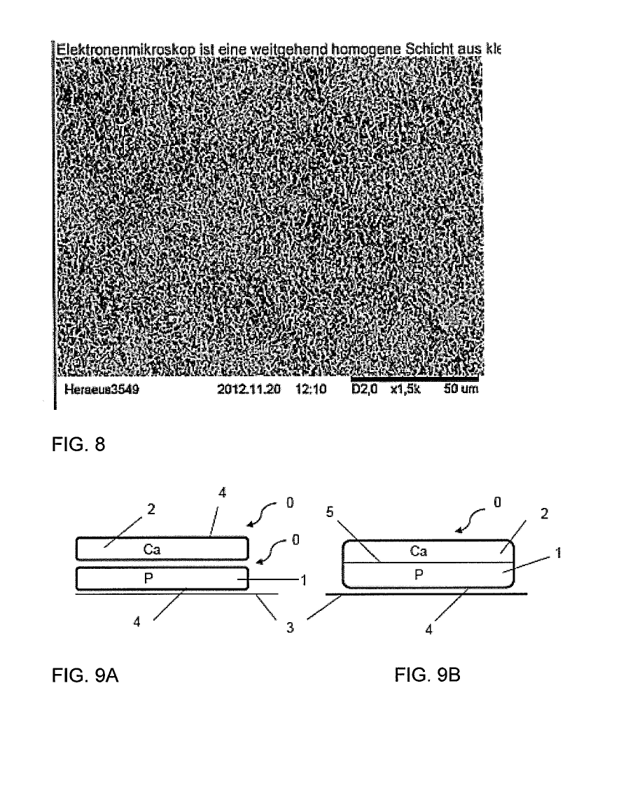

FIG. 8: Dentine surface showing growth. The pore-rich dentine surface is covered by a growth of a homogeneous layer of needle-like crystals. After treatment with the mineralization kit (GDA cross-linking on both sides).

1. A formulation comprising at least one partially elastic shaped body, said body comprising at least one mineralization matrix containing at least one gel, whereby the shaped body comprises, at least in part, in at least one plane a reduced solubility with respect to aqueous media as compared to the mineralization matrix, whereby the plane acts as membrane, and
a) the at least one mineralization matrix comprises a gel containing water-soluble phosphates or hydrolysable phosphates that form water-soluble phosphate ions and has a pH value of 2 to 8, and
b) the at least one or a second mineralization matrix comprises a second gel comprising calcium ions or compounds releasing calcium ions and has a pH value of 3.5 to 14.

2. Formulation according to claim 1,
wherein the at least one mineralization matrix is present in a first shaped body and the second mineralization matrix is present in a second shaped body.

3. Formulation according to claim 1 wherein
a) the at least one mineralization matrix comprises a gel comprising
(i) water-soluble phosphates or hydrolysable phosphates that form water-soluble phosphate ions,
(ii) a content of water or of a mixture of water and an organic solvent,
(iii) optionally, at least one carboxylic acid and/or a buffer system.

4. The formulation according to claim 1 wherein that the second mineralization matrix or the at least one mineralization matrix comprises, in (b) a second gel comprising
(i) calcium ions or compounds releasing calcium ions,
(ii) optionally, water or a mixture of water and an organic solvent, and
(iii) optionally, at least one carboxylic acid and/or a buffer system.

5. The formulation according to claim 1 wherein the at least one mineralization matrix comprises a gel comprising at least one water-soluble fluoride or one compound releasing fluorides.

6. The formulation according to claim 1 wherein the gel comprises at least one gel-forming agent selected from denatured collagen, hydrocolloids, polypeptides, protein—hydrolysis products, polysaccharides, polyacrylates or mixtures thereof.

7. The formulation according to claim 1 wherein the gel comprises gelatine and a polyol, the adducts thereof and/or the conversion products thereof.

8. The formulation according to claim 1 wherein the at least one partially elastic shaped body corresponds to the at least one mineralization matrix, whereby the mineralization matrix comprises in at least one at least partially chemically cross-linked plane a reduced solubility with respect to aqueous media as compared to the corresponding mineralization matrix that is not chemically cross-linked in this way.

9. The formulation according to claim 1 wherein the at least one mineralization matrix comprising at least one gel is present in at least one partially elastic shaped body in the form of a flat element or at least partial negative image of a jaw, whereby the shaped body comprises at least two planes that are arranged on the outer surface or comprises at least one partial outer envelope that comprises reduced solubility with respect to aqueous media as compared to the mineralization matrix.

10. The formulation according to claim 1 wherein the at least one or two mineralization matrices comprising at least one gel are present in the form of a flat element or at least partial negative image of a jaw and/or the at least one partially elastic shaped body is present in the form of a flat element or at least partial negative image of a jaw.

11. The formulation according to claim 1 wherein the planes or envelope form the outer boundary of the mineralization matrix.

12. The formulation according to claim 1 wherein the at least one plane or envelope is formed by chemical cross-linking of the mineralization matrix or by applying a coating onto the mineralization matrix in order to form an ion- and water-permeable surface of the mineralization matrix.

13. The formulation according to claim 1 wherein the chemical cross-linking in the at least one plane or for forming the envelope of the mineralization matrix comprising at least one gel is based on the chemical conversion products of gelatine or of a gelatine thermally stabilized by glycerol with a di- or polyfunctional cross-linker

14. The formulation according to claim 1 comprising:
(I) a shaped body in the form of a flat element having at least two separated mineralization matrices each in the form of a flat element containing the gel with the following layer structure in the shaped body:
a) a first mineralization matrix in the form of a flat element comprising gel and water-soluble phosphates or hydrolysable phosphates that form water-soluble phosphate ions, water-soluble fluorides or a compound releasing fluorides, water or a mixture of water and an organic solvent, optionally at least one carboxylic acid and/or a buffer system;
b) optionally membrane;
c) a second mineralization matrix in the form of a flat element comprising gel and calcium ions or compounds releasing calcium ions, optionally water or a mixture of water and an organic solvent, optionally at least one carboxylic acid and/or a buffer system;
whereby (I) the shaped body in the form of a flat element comprises at least one or two planes that are arranged on the outer surface or comprises an outer envelope which are (a), at least in part, obtinable by chemical cross-linking or (b) correspond to a coating, and which acts, in particular, as membrane, and comprise a reduced solubility with respect to aqueous media as compared to the mineralization matrices, or,
(II) two separate shaped bodies each independently in the form of a flat element having at least one mineralization matrix in the form of a flat element containing the gel, and the first shaped body comprises a first mineralization matrix in the form of a flat element comprising gel and and at least one water-soluble phosphate or hydrolysable phosphate that form water-soluble phosphate ions, and at least one water-soluble fluoride or compound releasing fluorides, water or a mixture of water and an organic solvent, optionally at least one carboxylic acid and/or a buffer system; and the
second shaped body comprises a second mineralization matrix in the form of a flat element comprising gel and at least one calcium ion or compound releasing calcium ions, optionally water or a mixture of water and an organic solvent, optionally at least one carboxylic acid and/or a buffer system;
whereby (II) the shaped bodies each independently comprise at least one or two planes that are arranged on the outer surface or an outer envelope, which (a) at least in part are obtainable, by chemical cross-linking or (b) correspond to a coating, and which act, in particular, as membrane and comprise a reduced solubility with respect to aqueous media as compared to the mineralization matrices.

15. The formulation according to claim 13 wherein the di- or polyfunctional cross-linker comprises dialdehydes, polyepoxides and/or polyisocyanates and mixtures comprising at least to cross-linkers.

16. The formulation according to claim 1 wherein the carboxylic acid is selected from fruit acids, amino acids, fatty acids, hydroxycarboxylic acids, dicarboxylic acids, and mixtures comprising at least two of the aforementioned acids and/or the buffer system comprises carboxylates of alkylcarboxylic acids, fatty acids, fruit acids, amino acids, hydroxycarboxylic acids, dicarboxylic acids, and mixtures thereof.

17. The formulation according to claim 1 comprising:
(I) a shaped body in the form of a flat element having at least two mineralization matrices, optionally separated by a membrane, containing the gel at a layer thickness of 50 to 6,000 μm, or (II) two separate shaped bodies each independently in the form of a flat element each having at least one mineralization matrix in the form of a flat element containing a gel, whereby each shaped body independently has a layer thickness of 10 to 3,000 μm, whereby the first shaped body comprising water-soluble phosphates or hydrolysable phosphates that form water-soluble phosphates has a layer thickness of 50 to 3,000 μm, and/or the second shaped body comprising calcium ions or compounds releasing calcium ions has a layer thickness of 10 to 3,000 μm.

18. Method for producing a formulation according to claim 1, suitable for depositing apatite selected from fluorapatite, hydroxylapatite or mixtures thereof on vertebrate teeth comprising
(I) producing a partially elastic shaped body comprising at least one mineralization matrix containing at least one gel containing water-soluble phosphates or hydrolysable phosphates that form water-soluble phosphate ions, whereby the shaped body comprises, at least in part, in at least one plane, a reduced solubility with respect to aqueous media as compared to the mineralization matrix, and producing the shaped body, wherein,
a) for producing at least one mineralization matrix containing the gel, in a first step, a mixture of
(i) 0.05 to 4 mol/l, in particular 0.5 to 1.5 mol/l water-soluble phosphates or hydrolysable phosphates that form water-soluble phosphate ions;
(ii) a corresponding amount of water or of a mixture of water and an organic solvent;
(iii) optionally at least one carboxylic acid and/or a buffer system;
(iv) 0 to 6,000 ppm by weight water-soluble fluoride or compound releasing fluorides is prepared, and using, in a further step, the mixture produced in a)
b) together with gelatine and optionally glycerol, while heating, to produce the gel;
c) forming of the gel, such that the mineralization matrix is formed, optionally solidification,
d) forming a plane arranged on the outer surface of the at least one mineralization matrix, while the shaped body is being formed.

19. Method for producing a formulation according to claim 1, suitable for biomimetic deposition of apatite selected from fluorapatite, hydroxylapatite or mixtures thereof on vertebrate teeth comprising
(I) producing at least one partially elastic shaped body comprising at least one mineralization matrix containing at least one gel containing calcium ions or compounds releasing calcium ions, whereby the shaped body comprises, at least in part, in at least one plane, a reduced solubility with respect to aqueous media as compared to the mineralization matrix, and producing the shaped body, wherein,
a) for producing at least one mineralization matrix containing the gel, in a first step, a mixture of
(i) 0.1 to 2 mol/l calcium ions or compounds releasing calcium ions;
(ii) a corresponding amount of water or of a mixture of water and an organic solvent;
(iii) optionally, at least one carboxylic acid and/or a buffer system is prepared; and using, in a further step, the mixture produced in a)
b) together with gelatine and optionally glycerol, while heating, to produce the gel;
c) forming of the gel, such that the mineralization matrix is formed, optionally solidification,
d) forming a plane arranged on the outer surface of the at least one mineralization matrix, while the shaped body is being formed.

20. Method according to claim 18, wherein in b), 5 to 50% by weight gelatine with and 0 to 30% by weight glycerol with respect to the total composition of the gel are added in a further step, preferably 25 to 40% by weight gelatine and 5 to 20% by weight glycerol are added to produce the matrix containing water-soluble phosphates or hydrolysable phosphates that form water-soluble phosphate ions, and 20 to 40% by weight gelatine and 15 to 25% by weight glycerol are added to produce the mineralization matrix containing calcium ions or compounds releasing calcium ions.

21. Method according to claims 18 to 20, characterized in that the gel produced in further step b) is being formed, in particular into a flat element or an individual three-dimensional element, and in that the gel can be solidified in said shape.

22. Method according to claim 21, wherein at least one plane of the at least one mineralization matrix that is arranged on the outer surface in the form of a flat element or at least partial negative image of a jaw, is contacted, in a further step, to a mixture containing a di- or polyfunctional cross-linker comprising dialdehydes, polyepoxides, polyisocyanates, and at least partial chemical cross-linking in at least one plane of the flat element that is arranged on the outer surface is produced while obtaining the at least one shaped body comprising at least one plane comprising a reduced solubility with respect to aqueous media as compared to the mineralization matrix; according to an alternative embodiment, all planes of the at least one flat element arranged on the outer surfaces are cross-linked in order to produce a cross-linked envelope of the at least one mineralization matrix while obtaining the at least one shaped body.

23. Method according to claim 18 wherein at least one mineralization matrix in the form of a flat element or at least partial negative image of a jaw is cross-linked in order to produce a shaped body containing phosphates and fluorides while producing an at least partial envelope and/or at least one mineralization matrix in the form of a flat element or at least partial negative image of a jaw is cross-linked in order to produce a shaped body containing calcium ions while producing an at least partial envelope.

24. Kit comprising at least one formulation comprising a partially elastic shaped body A and a separate partially elastic shaped body B, each independently comprising a formulation according to claim 1, whereby
(a) partially elastic shaped body A comprises
(a1) at least one mineralization matrix comprising at least one gel,
(a2) at least one water-soluble phosphate or hydrolysable phosphates that form water-soluble phosphate ions, and
(a3) optionally, at least one carboxylic acid and/or a buffer system
(a4) optionally, water-soluble fluorides or a compound releasing fluorides
(a5) optionally, a content of water or of a mixture of water and an organic solvent
(b) partially elastic shaped body B comprises
(b1) at least one mineralization matrix comprising at least one gel,
(b2) water-soluble calcium ions or compounds releasing calcium ions, and
(b3) optionally, at least one carboxylic acid and/or a buffer system
(b4) optionally, water or a mixture of water and an organic solvent, or a partially elastic shaped body C comprising a formulation, whereby the
(c) partially elastic shaped body C comprises
(c1) at least one mineralization matrix comprising at least one gel,
(c1.1) at least one water-soluble phosphate or hydrolysable phosphates that form water-soluble phosphate ions, and
(c1.2) optionally, at least one carboxylic acid and/or a buffer system
(c1.3) optionally, water-soluble fluorides or a compound releasing fluorides
(c1.4) optionally, a content of water or of a mixture of water and an organic solvent
(c2) optionally, a membrane (layer)
(c3) at least one mineralization matrix comprising at least one gel,
(c3.1) water-soluble calcium ions or compounds releasing calcium ions, and
(c3.2) optionally, at least one carboxylic acid and/or a buffer system
(c3.3) optionally, water or a mixture of water and an organic solvent, whereby the layer structure of the partially elastic shaped body C is c1 and c3 or c1, c2, and c3.

25. Cross-linker solution for use in the production of a formulation according to claim 1 wherein a phosphate mixture is produced by mixing
(i) 0.05 to 4 mol/1, 0.5 to 1.5 mol/l water-soluble phosphates or hydrolysable phosphates that form water-soluble phosphate ions, (ii) a corresponding amount of water or of a mixture of water and an organic solvent, (iii), optionally, at least one carboxylic acid and/or a buffer system, (iv) 0 to 6,000 ppm by weight water-soluble fluoride or a compound releasing fluoride, or in that
b) a calcium mixture is produced by mixing (i) 0.1 to 2 mol/l calcium ions or compounds releasing calcium ions, (ii) a corresponding amount of water or of a mixture of water and an organic solvent, (iii), optionally, at least one carboxylic acid and/or a buffer system, and mixing (a) and/or (b) with a defined amount of a solution containing a di- or polyfunctional cross-linker comprising dialdehydes, polyepoxides, polyisocyanates, whereby the cross-linker, in particular, is glutardialdehyde.

26. (canceled)
